(19) United States
(12) Patent Application Publication
(10) Pub. No.: US 2016/0192890 A1
(43) Pub. Date: Jul. 7, 2016

(54) METHODS AND SYSTEM FOR MONITORING PHYSICAL ACTIVITIES

(71) Applicant: Cardiac Pacemakers, Inc., St. Paul, MN (US)

(72) Inventors: Viktoria A. Averina, Shoreview, MN (US); Pramodsingh Hirasingh Thakur, Woodbury, MN (US); Qi An, Blaine, MN (US); Barun Maskara, Blaine, MN (US)

(21) Appl. No.: 14/984,990

(22) Filed: Dec. 30, 2015

Related U.S. Application Data
(60) Provisional application No. 62/099,281, filed on Jan. 2, 2015.

(51) Int. Cl. A61B 5/00; A61B 5/024; A61B 5/053; A61B 5/145; A61B 5/08; A61B 5/087; A61B 5/11; A61B 5/021; A61B 5/085

(52) U.S. Cl. CPC A61B 5/7282; A61B 5/021; A61B 5/024; A61B 5/053; A61B 5/085; A61B 5/0816; A61B 5/0826; A61B 5/087; A61B 5/1118; A61B 5/14542

(57) ABSTRACT

Systems and methods for monitoring physical activities or exercise are disclosed. A system can comprise an information receiver circuit capable of receiving information indicative of physical activities, and a physical activity analyzer circuit coupled to the information receiver circuit. The physical activity analyzer circuit can detect one or more activity parameters from the physical activity information, and classify the physical activity into one of a plurality of activity levels including a vigorous exercise, a moderate exercise, or a mild exercise or activities of daily living. The one or more activity parameters can include an activity intensity parameter, an activity duration parameter, or an activity transition pattern including a change or a rate of change from a first activity level to a different second activity level. The system can optionally include a heart failure detector that detects a HF event indicative of worsening HF using the activity levels.

### CLAIM OF PRIORITY

This application claims the benefit of priority under 35 U.S.C. §119(e) of U.S. Provisional Patent Application Ser. No. 62/099,281, filed on Jan. 2, 2015, which is herein incorporated by reference in its entirety.

### TECHNICAL FIELD

This document relates generally to electronic health-status monitoring system, and more particularly a system and method for monitoring exercise intensity of patients.

### BACKGROUND

Congestive heart failure (CHF) is a leading cause of death in the United States affecting approximately 670,000 individuals. CHF occurs when the heart is unable to adequately supply enough blood to maintain a healthy physiological state. CHF can be treated by drug therapy, or by implantable medical devices such as for providing cardiac resynchronization therapy (CRT). A patient's chronic stable heart failure may abruptly decompensate, requiring hospitalization.

Due to the prevalence of CHF related issues, it is prudent to seek out methodologies that would facilitate the prevention, monitoring, and treatment of heart disease on a daily basis. For example, monitoring a patient's CHF status can help avoid acute decompensation and hospitalization.

### OVERVIEW

Patients with CHF exhibit abnormal kinetics of physiological responses to activity (PRA). This can be demonstrated in a laboratory setting, such as by having a patient first maintain a low level of physical activity, until physiological processes become reasonably steady over time. Then, if an abrupt increase in activity is imposed, such as by placing the patient on a treadmill, for example, some physiological responses such as contractility or oxygen uptake efficiency may have an insufficient response in a CHF patient, as compared to a person without CHF. Other physiological responses, such as heart rate and respiratory rate, may be exaggerated in a CHF patient, as compared to a person without CHF. The abnormal kinetics of PRA can be related to the reduced cardiac function of the CHF patient, and contribute to patient symptoms such as shortness of breath, exercise limitation, or fatigue. The abnormal kinetics of PRA can be roughly proportional to the severity of cardiac limitation.

The present inventors have recognized, among other things, a need for improved diagnostic indicators, such as for diagnosing CHF status. Evaluation of physiological responses to activity in CHF patients may require continuous monitoring and effective assessment of rigorousness of physical activity or exercise capacity. Activity intensity or exercise capacity can provide indications of a subject's general health status. It can also be used for diagnostic purpose, such as for evaluating cardiovascular disease. Monitoring the activity or exercise level can also be beneficial in assessing benefit or efficacy of a HF therapy. For example, the CRT therapy can result in a large and long-term sustained improvement in a patient's capacity of physical activity. Such an increased exercise capacity is accompanied by increase in oxygen consumption, improved cardiac performance, reduced mitral regurgitation, and reduced sympathetic drive to the heart.

Regular monitoring of physical activity or exercise capacity can also provide a feedback to healthcare professionals on the efficacy of a therapy or medical treatment regimes, such as CRT therapy to improve cardiac performance in CHF patients. Additionally, physical activity information can also be used to adjust CHF therapy, such as by adapting cardiac pacing rate to various activity levels.

This document discusses, among other things, a system for monitoring physical activities or exercise. The system can receive information about physical activities, detect one or more activity parameters from the physical activity information, and classify the physical activity into one of a plurality of activity levels The system can optionally include a heart failure detector that detects a HF event indicative of worsening HF using the activity levels.

In Example 1, a system can comprise a physical activity information receiver circuit and a physical activity analyzer circuit coupled to the physical activity information receiver circuit. The physical activity information receiver circuit can receive information indicative of physical activity. The physical activity analyzer circuit can detect one or more activity parameters using the physical activity information, and can classify the physical activity into one of a plurality of activity levels using the one or more activity parameters. The one or more activity parameters can include an activity intensity parameter, an activity duration parameter, or an activity transition pattern including a change or a rate of change from a first activity level to a different second activity level. The plurality of activity levels can include two or more categorical activity levels.

In Example 2, the plurality of activity levels in Example 1 can include two or more of a vigorous exercise, a moderate exercise, or a mild exercise or activities of daily living, and the physical activity analyzer circuit of Example 1 can classify the physical activity as the vigorous exercise when the detected activity intensity parameter is within a first intensity zone and the activity duration parameter is within a first duration range. The physical activity analyzer circuit of Example 1 can classify the physical activity as the moderate exercise when the detected activity intensity parameter is within a second intensity zone and the activity duration parameter is within a second duration range. The first intensity zone can include activity intensity higher than that included in the second intensity zone, or the first duration range can include activity duration longer than that included in the second duration range.

In Example 3, the plurality of activity levels in Example 1 can include two or more of a vigorous exercise, a moderate exercise, or a mild exercise or activities of daily living, and the physical activity analyzer circuit of Example 1 can classify the physical activity as the vigorous exercise when the detected activity intensity parameter is within a first intensity zone, the activity duration parameter is within a first duration range, and the activity transition pattern can include a first rate of change of activity intensity. Alternatively, the physical activity analyzer circuit can classify the physical activity as the moderate exercise when the detected activity intensity parameter is within a second intensity zone, the activity duration parameter is within a second duration range, and the activity transition pattern can include a second rate of change of activity intensity. The first intensity zone can include activity intensity higher than that included in the second intensity zone, or the first duration range can include activity duration longer than that included in the second duration range, or the first rate of change can be greater than the second rate of change.

In Example 4, the system of any one of Examples 1 through 3 can comprise an activity categorizer circuit that can be configured to categorize the detected one or more activity parameters into one of a plurality of activity bins. An individual bin can be characterized by a respective activity intensity zone and a respective activity duration range, a higher activity bin having higher activity intensity or longer activity duration than a lower activity bin. The physical activity analyzer circuit can be configured to classify the physical activity using the categorization of the activity parameter and a bin-activity level association that corresponds the plurality of activity bins to one or more of the plurality of activity levels.

In Example 5, the physical activity information of Example 4 can include multiple activity episodes obtained during a specified period of time. The activity categorizer circuit can be further configured to generate an activity bin distribution using categorization of the multiple activity episodes, and determine at least one characteristic feature from the activity bin distribution. An individual bin can include a bin count indicating number of activity episodes categorized into the respective bin. The physical activity analyzer circuit can classify the physical activity using the at least one characteristic feature of the activity bin distribution.

In Example 6, the activity categorizer circuit of Example 5 can determine the at least one characteristic feature including a highest activity bin and a bin distribution pattern. The highest activity bin can include an activity episode of highest activity intensity among the multiple activity episodes. The bin distribution pattern can indicate a comparison of bin counts of the plurality of activity bins. The physical activity analyzer circuit can classify the physical activity as the vigorous exercise when the highest activity bin exceeds a first bin threshold, or classify the physical activity as the moderate exercise when (1) the highest activity bin is between the first bin threshold and a second bin threshold lower than the first bin threshold, and (2) the bin distribution pattern indicates the highest activity bin preceded by a specified number of lower activity bins with respective bin count below a specified bin count threshold value.

In Example 7, the system of any one of Examples 1 through 6 can further comprise a physiologic signal receiver circuit that can be configured to receive at least one physiologic signal obtained during the physical activity. The physical activity analyzer circuit can be to classify the physical activity using the one or more activity parameters and the at least one physiologic signal.

In Example 8, the physiologic signal receiver circuit of Example 7 can receive at least one of a respiratory signal or a cardiac hemodynamic signal.

In Example 9, the system of any one of Examples 1 through 8 can further comprise an adjudication input circuit that can be configured to receive adjudication or confirmation of the classified activity level. The physical activity analyzer circuit can be configured to classify the physical activity at least using the received adjudication.

In Example 10, the system of any one of Examples 1 through 9 can further comprise a heart failure (HF) detector circuit that can be configured to determine an activity trend indicative of temporal variation of the classified activity level, and to detect a HF event indicative of worsening HF at least using the activity trend.

In Example 11, the HF detector circuit of the Example 10 can operate in a first mode to detect the HF event in response to the physical activity being classified as a first activity level, or operate in a different second mode to detect the HF event in response to the physical activity being classified as a second activity level more vigorous than the first activity level. The HF detector, when operated in the first mode, can have a higher sensitivity in detecting historical HF events than when operated in the second mode.

In Example 12, the system of Example 10 can comprise a physiologic sensor circuit that can sense at least one physiologic signal obtained during the physical activity. The HF detector circuit can detect the HF event using both the activity trend and the sensed physiologic signal.

In Example 13, the system of any one of Examples 1 through 12 can comprise a heart failure (HF) stratifier circuit that can be configured to determine an exercise frequency of the detected categorical activity level during a specified time, and determine a likelihood indication of a future event of worsening HF. The likelihood indication can be inversely proportional to the exercise frequency.

In Example 14, the system of any one of Examples 1 through 13 can comprise an output circuit that can be configured to generate a human-perceptible presentation of information including the classified activity level and the detected one or more activity parameters.

In Example 15, the system of any one of Examples 1 through 14 can comprise an accelerometer coupled to the physical activity information receiver circuit, the accelerometer can sense an acceleration signal indicative of physical activity.

In Example 16, a method for analyzing physical activity experienced by a patient can comprise receiving information of physical activity, and detecting one or more activity parameters using the physical activity information. The one or more activity parameters can include an activity intensity parameter, an activity duration parameter, or an activity transition pattern including a change or a rate of change from a first activity level to a different second activity level. The method can include classifying the physical activity into one of a plurality of activity levels using the one or more activity parameters. The plurality of activity levels can include two or more categorical activity levels.

In Example 17, the plurality of activity levels in Example 16 can include two or more of a vigorous exercise, a moderate exercise, or a mild exercise or activities of daily living, and the method of classifying the physical activity of Example 16 can include classifying the physical activity as the vigorous exercise when the detected activity intensity parameter is within a first intensity zone, the activity duration parameter is within a first duration range, and the activity transition pattern includes a first rate of change of activity intensity meeting a specified criterion. The method of classifying the physical activity of Example 16 can alternatively include classifying the physical activity as the moderate exercise when the detected activity intensity parameter is within a second intensity zone, and the activity duration parameter is within a second duration range. The first intensity zone can have higher intensity than the second intensity zone, and the first duration range can have longer duration than the second duration range.

In Example 18, the method of Example 17 can further comprise categorizing the detected one or more activity parameters into one of a plurality of activity bins, and providing an activity bin-activity level association that corresponds the plurality of activity bins to one or more of the plurality of activity levels. An individual bin can be characterized by a respective activity intensity zone and a respective activity duration range, a higher activity bin having higher activity intensity or longer activity duration than a lower activity bin. The classifying the physical activity can include classifying the physical activity using the categorization of the activity parameter and the bin-activity level association.

In Example 19, the method of Example 18 can further comprise generating an activity bin distribution using categorization of multiple activity episodes obtained during a specified period of time, and determining at least one characteristic feature from the activity bin distribution. An individual bin can include a bin count indicating number of activity episodes categorized into the respective bin. The classifying the physical activity can include classifying the physical activity using the at least one characteristic feature of the activity bin distribution.

In Example 20, the method of Example 16 can further comprise receiving at least one physiologic signal obtained during the physical activity. At least one physiologic signal can include a respiratory signal or a cardiac hemodynamic signal. The classifying the physical activity can include classifying the physical activity using the one or more activity parameters and the at least one physiologic signal.

In Example 21, the method of Example 16 can further comprise determining an activity trend indicative of temporal variation of the classified activity level, and detecting a heart failure (HF) event indicative of worsening HF at least using the activity trend.

In Example 22, the method of Example 21 can further comprise receiving at least one physiologic signal obtained during the physical activity. The detecting the HF event can include detecting the HF event when the detected activity trend and the at least one physiologic signal meet respective criteria.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a Cardiac Rhythm Management (CRM) system and portions of an environment in which the CRM system can operate.

FIG. 2 illustrates an example of a physical activity detection and analyzer system.

FIG. 3 illustrates an example of a physical activity analyzer system.

FIG. 4 illustrates an example of an activity level decision circuit.

FIG. 5 illustrates an example of a heart failure (HF) event detection system using at least physical level information.

FIG. 6 illustrates an example of a method for analyzing physical activity.

FIG. 7 illustrates an example of a method for classifying physical activity based on categorization of physical activities.

FIG. 8 illustrates an example of a method for detecting a heart failure (HF) event using at least physical level information.

### DETAILED DESCRIPTION

Disclosed herein are systems, devices, and methods for monitoring physical activities or exercise. The physical activity information, such as sensed using an activity sensor, can be used to generate activity parameters including one or more of an activity intensity parameter, an activity duration parameter, or an activity transition pattern. The physical activity can be classified into one of a plurality of activity levels. The classification of physical activities or exercise can be used for detecting events indicative of worsening HF.

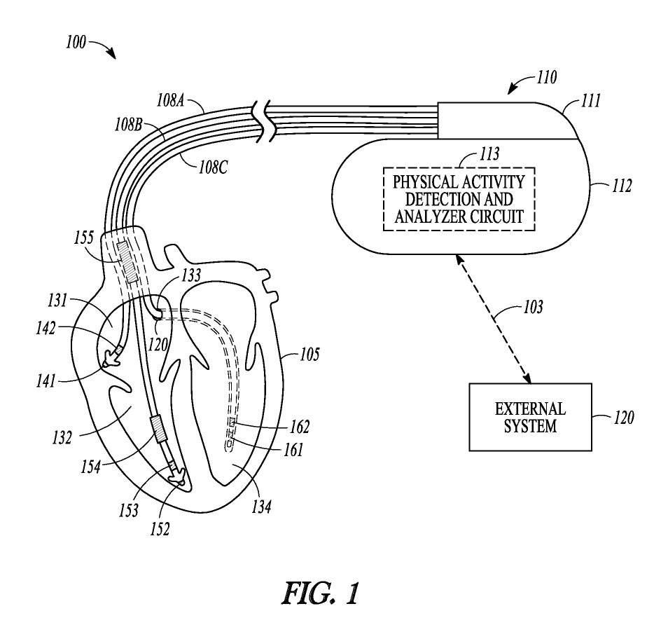

FIG. 1 illustrates an example of a Cardiac Rhythm Management (CRM) system 100 and portions of an environment in which the CRM system 100 can operate. The CRM system 100 can include an ambulatory medical device, such as an implantable medical device (IMD) 110 that can be electrically coupled to a heart 105 such as through one or more leads 108A-C, and an external system 120 that can communicate with the IMD 110 such as via a communication link 103. The IMD 110 may include an implantable cardiac device such as a pacemaker, an implantable cardioverter-defibrillator (ICD), or a cardiac resynchronization therapy defibrillator (CRT-D). The IMD 110 can include one or more monitoring or therapeutic devices such as a subcutaneously implanted device, a wearable external device, a neural stimulator, a drug delivery device, a biological therapy device, a diagnostic device, or one or more other ambulatory medical devices. The IMD 110 may be coupled to, or may be substituted by a monitoring medical device such as a bedside or other external monitor.

As illustrated in FIG. 1, the IMD 110 can include a hermetically sealed can 112 that can house an electronic circuit that can sense a physiological signal in the heart 105 and can deliver one or more therapeutic electrical pulses to a target region, such as in the heart, such as through one or more leads 108A-C. The CRM system 100 can include only one lead such as 108B, or can include two leads such as 108A and 108B.

The lead 108A can include a proximal end that can be configured to be connected to IMD 110 and a distal end that can be configured to be placed at a target location such as in the right atrium (RA) 131 of the heart 105. The lead 108A can have a first pacing-sensing electrode 141 that can be located at or near its distal end, and a second pacing-sensing electrode 142 that can be located at or near the electrode 141. The electrodes 141 and 142 can be electrically connected to the IMD 110 such as via separate conductors in the lead 108A, such as to allow for sensing of the right atrial activity and optional delivery of atrial pacing pulses. The lead 108B can be a defibrillation lead that can include a proximal end that can be connected to IMD 110 and a distal end that can be placed at a target location such as in the right ventricle (RV) 132 of heart 105. The lead 108B can have a first pacing-sensing electrode 152 that can be located at distal end, a second pacing-sensing electrode 153 that can be located near the electrode 152, a first defibrillation coil electrode 154 that can be located near the electrode 153, and a second defibrillation coil electrode 155 that can be located at a distance from the distal end such as for superior vena cava (SVC) placement. The electrodes 152 through 155 can be electrically connected to the IMD 110 such as via separate conductors in the lead 108B. The electrodes 152 and 153 can allow for sensing of a ventricular electrogram and can optionally allow delivery of one or more ventricular pacing pulses, and electrodes 154 and 155 can allow for delivery of one or more ventricular cardioversion/defibrillation pulses. In an example, the lead 108B can include only three electrodes 152, 154 and 155. The electrodes 152 and 154 can be used for sensing or delivery of one or more ventricular pacing pulses, and the electrodes 154 and 155 can be used for delivery of one or more ventricular cardioversion or defibrillation pulses. The lead 108C can include a proximal end that can be connected to the IMD 110 and a distal end that can be configured to be placed at a target location such as in a left ventricle (LV) 134 of the heart 105. The lead 108C may be implanted through the coronary sinus 133 and may be placed in a coronary vein over the LV such as to allow for delivery of one or more pacing pulses to the LV. The lead 108C can include an electrode 161 that can be located at a distal end of the lead 108C and another electrode 162 that can be located near the electrode 161. The electrodes 161 and 162 can be electrically connected to the IMD 110 such as via separate conductors in the lead 108C such as to allow for sensing of the LV electrogram and optionally allow delivery of one or more resynchronization pacing pulses from the LV. In an example, at least one of the leads 108A-C, or an additional lead other than the leads 108A-C, can be implanted under the skin surface without being within a heart chamber, or at or close to heart tissue.

The IMD 110 can include an electronic circuit that can sense a physiological signal. The physiological signal can include an electrogram or a signal representing mechanical function of the heart 105. The hermetically sealed can 112 may function as an electrode such as for sensing or pulse delivery. For example, an electrode from one or more of the leads 108A-C may be used together with the can 112 such as for unipolar sensing of an electrogram or for delivering one or more pacing pulses. A defibrillation electrode from the lead 108B may be used together with the can 112 such as for delivering one or more cardioversion/defibrillation pulses. In an example, the IMD 110 can sense impedance such as between electrodes located on one or more of the leads 108A-C or the can 112. The IMD 110 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 110 can be configured to inject current between an electrode on the RV lead 108B and the can housing 112, and to sense the resultant voltage between the same electrodes or between a different electrode on the RV lead 108B and the can housing 112. A physiologic signal can be sensed from one or more physiological sensors that can be integrated within the IMD 110. The IMD 110 can also be configured to sense a physiological signal from one or more external physiologic sensors or one or more external electrodes that can be coupled to the IMD 110. Examples of the physiological signal can include one or more of electrocardiogram, intracardiac electrogram, arrhythmia, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are possible.

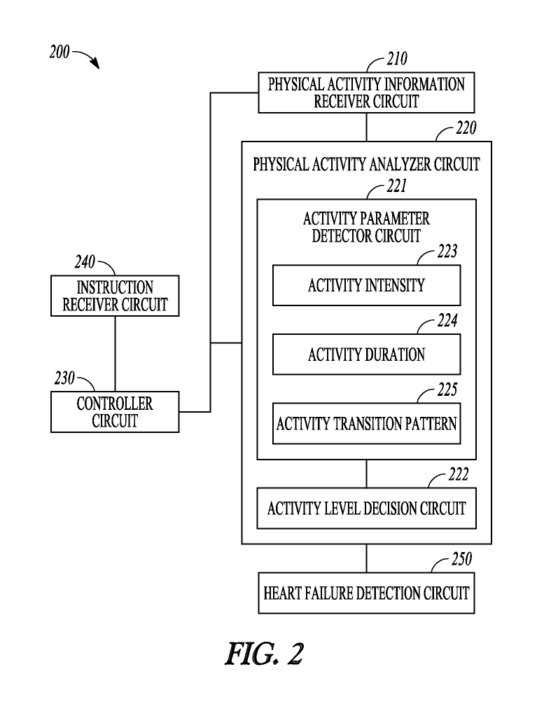

As illustrated, the CRM system 100 can include a physical activity detection and analyzer circuit 113. The physical activity detection and analyzer circuit 113 can be configured to detect one or more activity parameters from physical activity or exercise information, and to classify the physical activity into one of a plurality of activity levels using the one or more activity parameters. The physical activity or exercise information can include aerobic exercise, anaerobic exercise, or other intentional or sustained exercises. Examples of the plurality of activity levels can include two or more categorical activity levels such as two or more of a vigorous exercise, a moderate exercise, or a mild exercise or activities of daily living. In an example, the CRM system 100 can include a heart failure (HF) event detector circuit coupled to the physical activity detection and analyzer circuit 11. The HF event detector can be configured to detect an event indicative of worsening HF, such as a HF decompensation event. Examples of the physical activity detection and analyzer circuit 113 are described below, such as with reference to FIGS. 2-5.

The external system 120 can allow for programming of the IMD 110 and can receive information about one or more signals acquired by IMD 110, such as can be received via a communication link 103. The external system 120 can include a local external IMD programmer. The external system 120 can include a remote patient management system that can monitor patient status or adjust one or more therapies such as from a remote location.

The communication link 103 can include one or more of an inductive telemetry link, a radio-frequency telemetry link, or a telecommunication link, such as an internet connection. The communication link 103 can provide for data transmission between the IMD 110 and the external system 120. The transmitted data can include, for example, real-time physiological data acquired by the IMD 110, physiological data acquired by and stored in the IMD 110, therapy history data or data indicating IMD operational status stored in the IMD 110, one or more programming instructions to the IMD 110 such as to configure the IMD 110 to perform one or more actions that can include physiological data acquisition such as using programmably specifiable sensing electrodes and configuration, device self-diagnostic test, or delivery of one or more therapies.

The physical activity detection and analyzer circuit 113 can be implemented at the external system 120 such as using data extracted from the IMD 110 or data stored in a memory within the external system 120. Portions of the physical activity detection and analyzer circuit 113 may be distributed between the IMD 110 and the external system 120.

Portions of the IMD 110 or the external system 120 can be implemented using hardware, software, or any combination of hardware and software. Portions of the IMD 110 or the external system 120 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 110, the CRM system 100 could include a subcutaneous medical device (e.g., subcutaneous ICD, subcutaneous diagnostic device), wearable medical devices (e.g., patch based sensing device), or other external medical devices.

FIG. 2 illustrates an example of a physical activity detection and analyzer system 200, which can be an embodiment of the physical activity detection and analyzer circuit 113. The physical activity detection and analyzer system 200 can include one or more of a physical activity information receiver circuit 210, a physical activity analyzer circuit 220, a controller circuit 230, and an instruction receiver circuit 240. Additionally, the physical activity detection and analyzer system 200 can optionally include a heart failure detector circuit 250.

The physical activity information receiver circuit 210 can be configured to receive information indicative of physical activity, exercise, or exertion. In an example, the activity receiver circuit 210 can receive physical activity information from a device capable of collecting or storing activity information, such as an external programmer, a memory, a data repository such as an electronic medical record system, or other devices. In an example, the physical activity information receiver circuit 210 can be coupled to an activity sensor configured to sense from a patient an indication of physical activity, exercise, or exertion. The activity sensor can be an implantable, wearable, or otherwise ambulatory sensor. The activity sensor can be external to the patient or implanted inside the body. In an example, the activity sensor can be included in at least one part of an implantable system, such as an implantable device, or a lead coupled to the implantable device. The activity sensor can also include a sensor interface circuit, configured to process the acceleration signal and provide a resulting physical activity signal.

In an example, the activity sensor can include a single-axis or multi-axis accelerometer configured to sense an acceleration signal of at least a portion of the subject's body. The strength of the acceleration signal can be indicative of the physical activity level. The accelerometer can also be used for other purposes, such as to sense the subject's posture, heart sounds, or other information available from an acceleration signal.

The physical activity signal can be indicative of physical exertion of a subject. In an example, the physical activity information receiver circuit 210 can be coupled to respiratory sensor configured to measure respiratory parameters correlative or indicative of respiratory exchange, i.e., oxygen uptake and carbon dioxide output. Examples of the respiratory parameters can include respiration rate, tidal volume, minute ventilation, peak or trough of a respiration signal, or other indicators of respiration depth; descriptors of respiration pattern such as apnea index indicating the frequency of sleep apnea, hypopnea index indicating the frequency of sleep hypopnea, apnea-hypopnea index (AHI) indicating the frequency of or sleep hypopnea events, or a rapid shallow breathing index (RSBI) computed as a ratio of respiratory frequency (number of breaths per minutes) to tidal volume, among other respiratory parameters.

In an example, two or more physiologic sensors can be combined to provide a composite index of physical activity or exercise, such as a combination of accelerometers, respiration sensor, heart rate sensors, blood oxygen saturation sensors, among others. The physical activity or exercise information provided by the combined sensors can include a patient's physiological response to activity (PRA), such as one or both of abnormal breathing and abnormal reflex sympathetic activation due to activity. Reference is made to commonly assigned Beck et. al. U.S. patent application Ser. No. 13/024,720, entitled “KINETICS OF PHYSIOLOGICAL RESPONSE TO ACTIVITY DURING ACTIVITIES OF DAILY LIVING,” filed Feb. 10, 2011 (Attorney Docket No. 279.H86US1), which is hereby incorporated by reference in its entirety.

The physical activity analyzer circuit 220, coupled to the physical activity information receiver circuit 210, can include an activity parameter detector circuit 221 and an activity level decision circuit 222. The physical activity analyzer circuit 220 can be implemented as a part of a microprocessor circuit in the physical activity detection and analyzer system 200. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the functions, methods, or techniques described herein. The physical activity analyzer circuit 220 can include a signal conditioning circuit that can process the received activity information such as an acceleration signal indicative of physical activity or exercise. The processing can include amplification, digitization, filtering, or other signal conditioning operations. In an example, the signal conditioning circuit can include a bandpass filter adapted to filter the acceleration signal to a frequency range of approximately between 0 and 10 Hz. In an example, the physical activity signal (such as the acceleration signal) can be compared to a specified threshold, and the number of times the physical activity signal crosses the specified threshold within a specified time can be determined. In an example, a portion of the physical activity signal can be selected based on a specified condition, such as when the intensity, change, or rate of change of the physical activity falls within a specified range.

The activity parameter detector circuit 221 can be configured to detect one or more activity parameters using the received physical activity information. Examples of the activity parameters can include an activity intensity parameter 223 indicative of vigorousness of an exercise or magnitude of exertion, an activity duration parameter 224 including time spent at, or within a particular margin of, a specified activity intensity, or an activity transition pattern 225. Examples of the activity transition pattern can include a change, a rate of change, or a pattern of transition from a first activity level to a different second activity level. In an example, the activity transition pattern 225 can include a transition pattern from a first lower-intensity activity to a second higher-intensity activity (i.e., “ramp-up” transition), or a transition from a first higher-intensity activity to a second lower-intensity activity (i.e., “ramp-down” transition). Other examples of activity parameters can include variability of activity intensity within a specified time period, activity frequency such as number of episodes at a particular activity level during a specified period of time (e.g., a week or a month), among other activity parameters.

The activity level decision circuit 222 can classify the physical activity or exercise into one of a plurality of activity levels using the one or more activity parameters such as produced by the activity parameter detector circuit 221. The plurality of activity levels can differ from one another in at least one activity parameters such as the activity intensity, the activity duration, the activity transition pattern, or any other descriptor of activity. In an example, the plurality of activity levels can include two or more categorical activity levels such as two or more of a vigorous exercise, a moderate exercise, or a mild exercise or activities of daily living. Examples of vigorous exercise can include running, fast swimming, jumping rope, dancing, or competitive sports including basketball, football, soccer, tennis, etc. Examples of moderate exercise can include brisk walking or jogging, recreational biking, general gardening, mopping floor, scrubbing the bathtub, etc. Examples of the mild exercise can include light walking, stretching, washing dishes, doing laundry, playing musical instrument, etc. In another example, the plurality of activity levels can include one or more of very light, light, moderate, hard, very hard, and maximal levels. In another example, the plurality of activity levels can be based on cardiopulmonary effort or exertion, and the activity levels can include one or more of little effort, warm-up or recovery effort, aerobic effort, and anaerobic effort.

In an example, classification of activity levels can be based on the activity intensity. The activity levels can differ from one another by non-identical intensity zones, such as non-overlapping intensity zones. The activity level decision circuit 222 can classify a physical activity into one of the plurality of activity levels using a comparison between the activity intensity and different intensity zones.

The classification of activity levels can be based on the activity intensity and the activity duration. In an example, the activity level decision circuit 222 can classify the physical activity as vigorous exercise when the detected activity intensity parameter is within a first intensity zone (I_{V}) and the activity duration parameter is within a first duration range (σ_{V}); or classify the physical activity as moderate exercise when the detected activity intensity parameter is within a second intensity zone (I_{M}) and the activity duration parameter is within a second duration range (σ_{M}). The first intensity zone I_{V} includes activity intensity higher than that included in the second intensity zone I_{M}, or the first duration range σV includes activity duration longer than that included in the second duration range σ_{M}. In an example where the activity intensity is measured using acceleration signal such as produced by an accelerometer sensor, the intensity zones and the corresponding activity durations can be determined as follows: (1) for vigorous exercise, I_{V} includes acceleration values equal to or greater than approximately 115 mG, and σ_{V} is approximately 20 minutes or longer; (2) for moderate exercise, I_{M} includes acceleration values approximately between 80 mG and 115 mG, and σ_{M} is approximately 20 minutes or longer. Detected activities with intensity less than 80 mG can be classified as mild exercise or activities of daily living.

In an example, classification of activity levels can be further based on the activity transition patterns. The activity level decision circuit 222 can classify the physical activity as vigorous exercise when the detected activity intensity parameter is within I_{V}, the activity duration parameter is within σ_{V}, and the activity transition pattern includes a first rate of change (δ_{V}) of activity intensity; or classify the physical activity as moderate exercise when the detected activity intensity parameter is within I_{M}, the activity duration parameter is within σ_{M}, and the activity transition pattern includes a second rate of change (δ_{M}). The vigorous exercise and moderate exercise differ by at least one of the intensity parameter, duration parameter, or activity transition pattern. For example, the first intensity zone I_{V} includes activity intensity higher than that in the second intensity zone I_{M}, or the first duration range σ_{V} includes activity duration longer than that included in the second duration range σ_{M}, or the first rate of change δ_{V} is greater than the second rate of change δ_{M}.

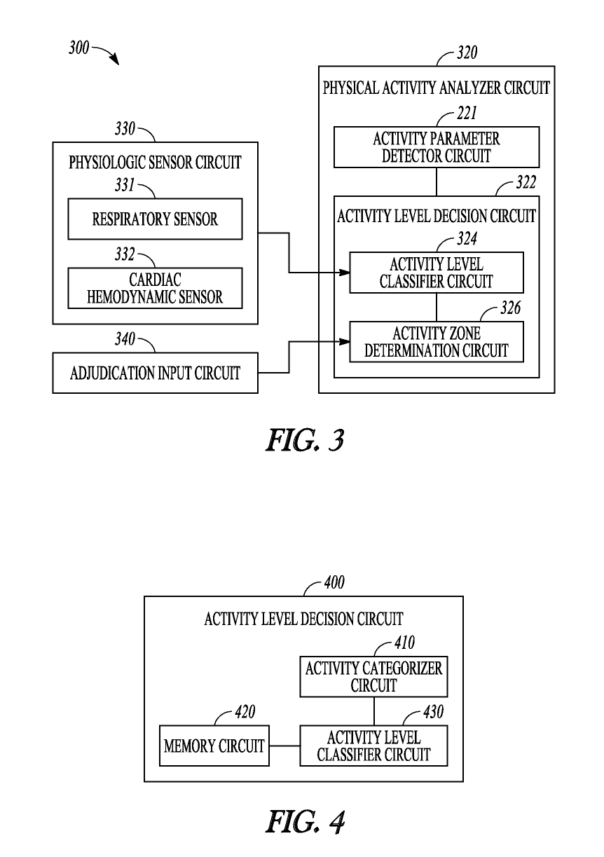

In various examples, the one or more activity parameters can be categorized into one of a plurality of activity bins such as having a pre-determined range of activity intensity. The physical activity analyzer circuit 220 can make the classification of activity levels using the activity bins. In another example, the physical activity analyzer circuit 220 can additionally include physiological sensors that can sense one or more physiologic signals during the sensed physical activity, and the activity level decision circuit 222 can be configured to classify the physical activity using both the activity parameters and the physiologic signals. Examples of variants of the physical activity analyzer circuit 220 are described below, such as with reference to FIGS. 3-4.

The classification of the physical activity, such as produced by the physical activity analyzer circuit 220, can be used in one or more ways. In an example, the physical activity detection and analyzer system 200 can include an output circuit configured to generate a human-perceptible presentation of the detected one or more activity parameters, the detected activity level, or the activity classification results, among other physical or physiologic information obtained from the subject. The human-perceptible presentation can also include a trend of activity levels or of other physiologic measurements, summaries or statistics produced using the historical activity levels or other physiologic measurements, or a comparison of the detected activity levels with a predetermined target exercise levels (e.g., exercise intensity, duration, frequency, etc.). The output circuit can deliver the presentation to a system user (e.g., a healthcare professional or a patient) such as via a user interface implemented in the external system 120. The presentation can include audio, text, graph, animation, or other audio-visual media formats that can inform, alert, or alarm the system user of the detected physical activity. In another example, the output circuit can include a transmitter module configured to transmit the classified activity level and the detected one or more activity parameters, via a wired or wireless communication network, to a portable electronic device such as a handheld or wearable mobile communication device. The portable electronic device can receive the detected activity level information and generate human-perceptible presentation of the physical activity. Transmission of activity level information can be triggered automatically on a scheduled or periodic basis, such as every 10-30 minutes, every hour, every day, every week, every month, or at any specified period or frequency. Alternatively or additionally, the transmission can be triggered manually such as in response to a command signal provided by the system user such as via the external system 120, or in response to a specified event detected by the IMD 110.

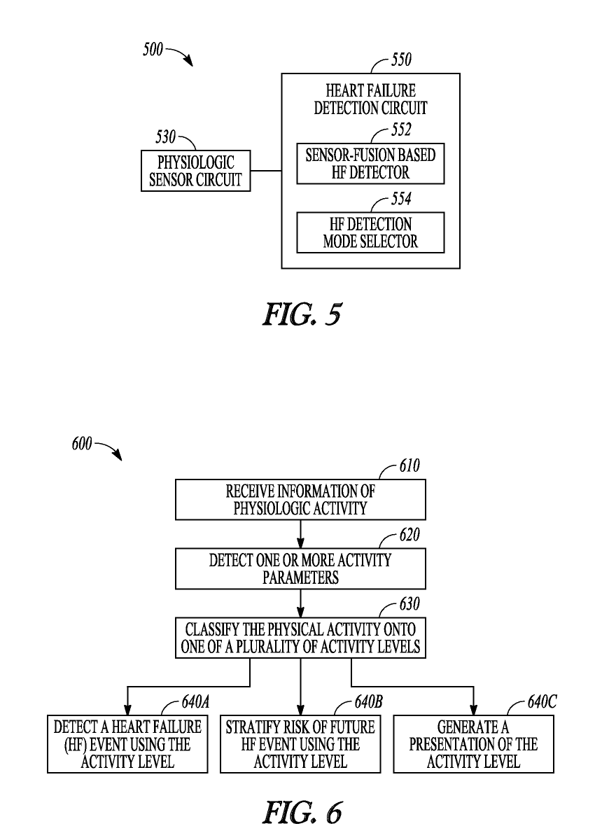

In an example, the physical activity detection and analyzer system 200 can be coupled to a diagnostic circuit. The diagnostic circuit can use the classified physical activities, either alone or in combination with other physiologic signals, to provide assessment of general health status, or patient diagnostic information, such as pulmonary edema, chronic obstructive pulmonary disease (COPD), asthma and pneumonia, myocardial infarction, dilated cardiomyopathy (DCM), ischemic cardiomyopathy, valvular disease, renal disease, peripheral vascular disease, cerebrovascular disease, hepatic disease, diabetes, anemia, depression, pulmonary hypertension, sleep disordered breathing, hyperlipidemia, among others. As illustrated in FIG. 2, the physical activity detection and analyzer system 200 can optionally include a heart failure (HF) detector circuit 250. The HF detector circuit 250 can be configured to determine an activity trend indicative of temporal variation of the classified activity level, and to detect a HF event indicative of worsening HF by using at least the activity trend. Examples of the detector circuit 250 are described below, such as with reference to FIG. 5.

In an example, the physical activity detection and analyzer system 200 can be coupled to a therapy delivery circuit. The therapy delivery circuit can deliver a therapy to a patient at least in response to the classified activity levels. In an example, one or more therapy parameters, such as rate, frequency, duration, duty cycle, pulse width, pulse amplitude, or other parameters of electrostimulation, can be adjusted based on the detected activity levels. The detected activity levels can also be used to adjust therapy control parameters. In an example, in response to a detection of vigorous exercise, one or more parameters controlling the mode or rate of cardiac pacing can be adjusted to better support increased metabolic demand during the exercise, such as by reducing the atrioventricular delay (AVD), increasing lower rate limit (LRL), or adjusting other device therapy control parameters.

In an example, the physical activity detection and analyzer system 200 can optionally include a HF risk stratifier circuit configured to compute a likelihood indication of a future event of worsening HF, such as a HF decompensation event in a specified timeframe (e.g., within approximately 1-3 months, 3-6 months, or beyond 6 months). In an example, exercise frequency of the detected categorical activity level, such as moderate or vigorous exercise (F_{M} and F_{V} respectively), can be measured as the number of episodes of respective activity levels within a specified time period, and the likelihood indication can be inversely proportional to the exercise frequency. In an example, a subject who has an F_{M} exceeding a specified frequency threshold (e.g., 6 episodes per month), or an F_{V} exceeding a specified frequency threshold (e.g., 3 episodes per month), is deemed to be at lower risk of developing future HF events than those whose F_{M} or F_{V} is below the specified frequency threshold. In various examples, additional physiologic signals can be used in HF risk stratification. Examples of the such physiologic signals can include one or more of electrocardiogram, intracardiac electrogram, arrhythmia, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, body temperature, among other physiologic signals.

The controller circuit 230 can receive external programming input from the instruction receiver circuit 240 to control the operations of the physical activity information receiver circuit 210 and the physical activity analyzer circuit 220, and the data flow and instructions between these components and respective subcomponents. Examples of the instructions received by instruction receiver 240 can include parameters used in sensing a activity signal from an activity sensor or processing received activity information, detecting activity parameters from the physical activity information, and classifying the physical activity into one of the plurality of activity levels. When an optional heart failure detector circuit 250, or other optional diagnostic, therapeutic, risk stratifier, or otherwise output circuit is included in the physical activity detection and analyzer system 200, the instruction receiver circuit 240 can also receive parameters for performing respective operations, such as parameters used in detecting HF event, parameters for stratifying the risk of future HF event, parameters for making diagnosis, or parameters of for delivering therapies. The instruction receiver circuit 240 can include a user interface configured to present programming options to a system user, and receive the system user's programming input. In an example, at least a portion of the instruction receiver circuit 250, such as the user interface, can be implemented in the external system 120.

FIG. 3 illustrates an example of a physical activity analyzer system 300, which can be an embodiment of at least a part of the physical activity detection and analyzer system 200. The physical activity analyzer system 300 can include a physical activity analyzer circuit 320, a physiologic sensor circuit 330, and an adjudication input circuit 340.

The physical activity analyzer circuit 320 can be an embodiment of the physical activity analyzer circuit 220, and include an activity parameter detector circuit 221 and an activity level decision circuit 322. The activity parameter detector circuit 221, as discussed above with reference to FIG. 2, can detect one or more activity parameters, including an activity intensity parameter, an activity duration parameter, an activity transition pattern, or a variability of activity intensity, among other activity parameters. The activity level decision circuit 322, which can be an embodiment of the activity level decision circuit 222, can include an activity level classifier circuit 324 and an activity zone determination circuit 326.

The activity level classifier circuit 324 can be coupled to a physiologic sensor circuit 330 which can sense one or more physiologic signals obtained during the physical activity. As illustrated in FIG. 3, the physiologic sensor circuit 330 can include one or both of a respiratory sensor 331 and a cardiac hemodynamic sensor 332.

The respiratory sensor 331 can include an impedance sensor, a thermocouple or thermistor-based air-flow sensor, or a piezo-resistive sensor, among other sensors that can directly or indirectly sense a respiration signal. The cardiac hemodynamic sensor 332 can include an implantable, wearable, or other ambulatory physiologic sensor that directly or indirectly measures dynamics of the blood flow in a heart chamber or in a blood vessel. Examples of the hemodynamic sensors can include heart rate sensor, a pressure sensor configured for sensing arterial pressure, pulmonary artery pressure, left atrial pressure, RV pressure, LV coronary pressure; impedance sensors configured for sensing thoracic impedance or cardiac impedance; a temperature sensor configured for sensing blood temperature; an accelerometer or a microphone configured for sensing one or more heart sounds; an optical sensor such as a pulse oximeter configured for sensing blood oxygen saturation; a chemical sensor configured for sensing central venous pH value, or oxygen or carbon dioxide level in the blood or other tissues or organs in the body.

One or more respiratory parameters or hemodynamic parameters can be derived respectively from the sensed respiratory signal or the sensed cardiac hemodynamic signal. Examples of the respiratory parameters can include a respiration rate, tidal volume, minute ventilation, peak or trough of a respiration signal, or other indicators of respiration depth; descriptors of respiration pattern such as apnea index indicating the frequency of sleep apnea, hypopnea index indicating the frequency of sleep hypopnea, apnea-hypopnea index (AHI) indicating the frequency of or sleep hypopnea events, or a rapid shallow breathing index (RSBI) computed as a ratio of respiratory frequency (number of breaths per minutes) to tidal volume. Examples of the hemodynamic parameters can include S1, S2, S3, or S4 heart sound components from the sensed heart sound signal, peak or trough impedance from the cardiac impedance signal, peak or trough blood pressure (corresponding respectively to systolic and diastolic pressures) from the blood pressure signal, or timing information associated with these signal components or characteristics.

The activity level classifier circuit 324 can use one or more activity parameters (such as produced by the activity parameter detector circuit 221) and at least one physiologic parameter (such as produced by the physiologic sensor circuit 330) to classify the physical activity into one of the plurality of activity levels. In an example, the activity level classifier circuit 324 can classify the physical activity as vigorous exercise when one or more detected activity intensity parameters fall within respective ranges (e.g., the activity intensity falls within a first intensity zone, or the activity duration parameter is within a first duration range), and at least one physiologic parameter meets a specified criterion (e.g., the heart rate (HR) exceeds a specified threshold or exceeds a baseline HR such as resting HR by a specified margin, or the respiration rate (RR) exceeds a specified threshold or exceeds a baseline RR such as resting RR by a specified margin). Likewise, the activity level classifier circuit 324 can classify the physical activity as moderate exercise when one or more detected activity intensity parameters fall within respective ranges different than those for vigorous exercise, and at least one physiologic parameter meets a specified criterion different than the criterion for vigorous exercise (e.g., a different HR threshold or RR threshold).

The activity zone determination circuit 326 can be configured to determine or adjust threshold values or zone ranges that define various activity levels, such as the intensity zones, activity duration ranges, or threshold for rate of change from one activity level to another activity level. The activity level classifier circuit 324 can be coupled to the activity zone determination circuit 326, and use the adjusted threshold values or zone ranges to classify the physical activity into different classes.

As illustrated in FIG. 3, the activity zone determination circuit 326 can be coupled to an adjudication input circuit 340. In an example, at least a portion of the adjudication input circuit 340 can be implemented in the instruction receive circuit 240. The activity zone determination circuit 326 can receive adjudication, confirmation, rejection, or other input about the classified activity level from a system user, and determine or adjust one or more of the thresholds or activity intensity zone ranges for one or more activity levels. In an example, the thresholds or activity intensity zone ranges can be adjusted using an adaptive process. For example, assuming a first activity level (e.g., vigorous exercise) and a second activity level (e.g., moderate exercise) differ at least by an intensity threshold TH_{I}, such that the two activity classes can be characterized at least by I_{V}>TH_{I} and I_{M}<TH_{I}. The activity zone determination circuit 326, using the adjudication input, can determine a lowest intensity among the episodes in the first activity level class (e.g., vigorous exercise with lowest intensity (minI_{V})), and a highest intensity among the episodes in the second activity level class (e.g., moderate exercise with highest intensity (maxI_{M})). The activity zone determination circuit 326 can then update TH_{I}, by computing a new TH_{I}′, using an optimal separation between minI_{V} and maxI_{M}, as follows: TH_{I}′=α*TH_{I}+β*(minI_{V}+maxI_{M})/2), where α and β are user-specified scalars that control the memory of the old threshold (via α) and the effect of adjudication-based optimal separation between the two activity classes (via β). In an example, 0≦α≦1, 0≦β≦1, and α+β=1.

FIG. 4 illustrates an example of an activity level decision circuit 400, which can be an embodiment of the activity level decision circuit 222, or the activity level decision circuit 322. The activity level decision circuit 400 can include an activity categorizer circuit 410, a memory circuit 420, and an activity level classifier circuit 430.

The activity categorizer circuit 410 can be configured to categorize the detected one or more activity parameters into one of a plurality of activity bins {βᵢ} for i=1, 2, . . . , K, where “i” denotes bin index, and K denotes the highest bin number corresponding to highest level of activity that can be sensed by the activity sensor. Each activity bin βᵢ can be characterized by a respective activity intensity zone, or a respective activity duration range. A higher activity bin can have higher activity intensity or longer activity duration than a lower activity bin. In an example, the activity intensity can be measured as acceleration sensed by an accelerometer sensor, and the activity bin βᵢ can be defined at least by a respective intensity range such as between Xᵢ mG and Yᵢ mG, where mG denotes unit of acceleration. A detected activity episode with intensity between Xᵢ mG and Yᵢ mG, and a corresponding activity duration exceeding a specified duration threshold (e.g., 10 minutes or 20 minutes), can be categorized into activity bin Bᵢ.

In an example, the activity categorizer circuit 410 can receive activity parameters associated with multiple activity episodes obtained during a specified period of time, such as during a day. In an example, one or more of the multiple activity episodes can be obtained from one exercise session, such as a voluntary aerobic exercise session. The exercise session can include various phases of exercises, including, for example, warming-up stretching, walking, jogging or power walking, running, sprinting, and recovery walk, etc. Each activity episode can be categorized into one of the plurality of bins at least based on the one or more activity parameters obtained from the respective activity episode. As such, an individual bin (Bᵢ) can include a bin count (NO indicating number of activity episodes categorized into the activity bin Bᵢ.

The activity categorizer circuit 410 can generate an activity bin distribution indicating spreading of the bin counts across M bins {Bᵢ} (for i=1, 2, . . . , M, and M≦K), where bin B_{M} is the highest activity bin that contains the activity episode with the highest activity level among the multiple activity episodes. In an example, the activity bin distribution can include a histogram of M activity bins. The activity categorizer circuit 410 can determine at least one characteristic feature from the activity bin distribution. In an example, the characteristic feature includes the bin index (M) of the highest activity bin B_{M}. In another example, the characteristic feature includes a bin distribution pattern indicative of relative bin counts across the plurality of activity bins.

The memory circuit 420 can be configured to store and maintain an activity bin—activity level association map. The activity bin—activity level association map can be constructed as a lookup table or other data structure, which establishes an association between an activity bin and an activity level (such as moderate or vigorous exercises) over a period of time, such as a day. In an example, activity bins at or above B₁₃ (which corresponds to acceleration approximately above 143 mG) can be mapped to vigorous exercise. Activity bins B₇ through B₁₂ (which correspond to a range of acceleration of approximately 66 mG to 132 mG) can be mapped to moderate exercise. Activity bins B₁ through B₆ (which correspond to acceleration of approximately below 66 mG) can be mapped to mild exercise or activities of daily living.

The activity level classifier circuit 430, coupled to the activity categorizer circuit 410 and the memory circuit 420, can be configured to classify the physical activity using the categorization of the activity parameter and the activity bin-activity level association map. Additionally or alternatively, the activity level classifier circuit 430 can use one or more of the characteristic features of the activity bin distribution to classify the physical activity. In an example, the activity level classifier circuit 430 can classify the physical activity as vigorous exercise when the highest activity bin B_{M} exceeds a bin threshold for vigorous exercise. An example of the bin threshold for vigorous exercise is B₁₃. As such, an activity intensity greater than 143 mG can be categorized into a bin higher than B₁₃, and can be classified as vigorous exercise. In another example, the activity level classifier circuit 430 can classify the physical activity as moderate exercise when (1) the highest activity bin B_{M} is between first and second bin thresholds (e.g., the first and second bin thresholds are B₇ and B₁₂, respectively), and (2) the bin distribution pattern indicates the highest activity bin is preceded by a specified number of lower activity bins with respective bin count below a specified bin count threshold value. The present inventors have recognized that a moderate exercise can have a characteristic activity transition pattern including an abrupt transition from a lower activity level to a higher activity level. The detected activity during the transition phase, even though meeting the intensity requirement of one or more “transitional” bins, can nevertheless be too short to meet the duration requirement of the “transitional” bins (e.g., 10 minutes), and is therefore not to be categorized into the “transitional” bins. For example, if the highest activity bin B_{M} is between B₇ and B₁₂, and the bin B_{M} is separated from lower bins by at least two bins with zero bin counts, it suggests that the “transitional” physical activity prior to B_{M} does not sustain long enough to be counted into one of the transitional bins. That is, the physical activity at issue is preceded by an abrupt transition from an earlier lower activity level. As such, the activity level classifier circuit 430 can classify the physical activity as moderate exercise.

FIG. 5 illustrates an example of a heart failure (HF) event detection system 500 using at least physical activity level information, which can be a part of the physical activity detection and analyzer system 200. The HF event detection system 500 can include a HF detector circuit 550 and a physiologic sensor circuit 530.

The physiologic sensor circuit 530 can sense one or more physiologic signals obtained during the physical activity. The physiologic sensor circuit 530 can be an embodiment of the physiologic sensor circuit 330. In an example, the physiologic sensor circuit 530 can include physiologic sensor signals different from those produced in the physiologic sensor circuit 330.

The HF detector circuit 550 can be an embodiment of the HF doctor circuit 250, and configured to detect a HF event indicative of worsening HF at least using an activity trend indicative of temporal variation of the classified activity level. The HF detector circuit 550 can include one or both of a sensor-fusion based HF detector 552, or a HF detection mode selector 554. The sensor-fusion based HF detector 552 can be configured to detect a HF event using both the activity trend and the sensed physiologic signal. In an example, the physiologic signals include one or more of a respiration rate (RR) signal, a tidal volume (TV) signal, or a heart rate (HR) signal. The sensor-fusion based HF detector 552 can detect a HF event in response to (1) the activity being classified as a lower level exercise (e.g., mild exercise or activity of daily living) and (2) the RR or TV exceeding their respective baseline value (RR₀ or TV₀, such as obtained during a resting state) by a specified margin. In another example, the sensor-fusion based HF detector 552 can detect an onset or worsening of a comorbidity condition of the HF, such as asthma or edema, in response to a moderate exercise accompanied by RR exceeding the baseline value RR₀ by a specified margin.

The HF detection mode selector 554 can be configured to select between two or more HF detection modes under which a HF event detector (such as the sensor-fusion based HF detector 552) can operate. For example, the HF event detector, when operated in the first mode (“high-sensitivity mode”), has a higher sensitivity in detecting historical HF events than when operated in the second mode (“low-sensitivity mode”). Examples of the high-sensitivity mode can include lower threshold for sensor signals in detecting HF event, or fewer sensors for detecting HF event. The present inventors have recognized that, compared to no exercise or lower-level exercise, higher level activities can be associated with lower risk of future worsening HF event such as a HF decompensation event. The HF detection mode selector 554 can select “high-sensitivity mode” for the HF event detector to detect a HF event in response to the physical activity being classified as a first mild or moderate activity level, or to select “low-sensitivity mode” in response to the physical activity being classified as a second more vigorous activity level.

FIG. 6 illustrates an example of a method 600 for analyzing physical activity. The method 600 can be implemented and operate in an implantable, wearable, or other ambulatory medical device, or in a remote patient management system. In an example, the method 600 can be performed by the physical activity detection and analyzer system 200 or any modification thereof.

The method 600 can being at step 610, where information of physical activity can be received, such as by using the physical activity detection and analyzer system 200. The activity information, indicative of physical activity, exercise, or exertion, can be sensed using an activity sensor. Examples of the activity sensor can include a single-axis or multi-axis accelerometer configured to sense acceleration of at least a portion of the subject's body, or a respiratory sensor configured to sense respiration effort indicative of physical exertion, among other sensors. In an example, the physical activity information can be received from a device capable of collecting or storing activity information, such as an external programmer, a memory, a data repository such as an electronic medical record system, or other devices.

At 620, one or more activity parameters can be detected from the received physical activity information. Examples of the activity parameters can include an activity intensity parameter indicative of vigorousness of an exercise or magnitude of exertion, an activity duration parameter including time spent at or within a particular margin of a specified activity intensity, or an activity transition pattern including a change or a rate of change from a first activity level to a different second activity level. Examples of the activity transition pattern can include a transition from a first lower-intensity activity to a second higher-intensity activity (i.e., “ramp-up” transition), or a transition from a first higher-intensity activity to a second lower-intensity activity (i.e., “ramp-down” transition).

At 630, the physical activity can be classified into one of a plurality of activity levels using the one or more activity parameters, such as produced at 630. The plurality of activity levels can include two or more categorical activity levels such as two or more of a vigorous exercise, a moderate exercise, or a mild exercise or activities of daily living. In another example, the plurality of activity levels can include one or more of very light, light, moderate, hard, very hard, and maximal levels. In an example, the plurality of activity levels can be based on cardiopulmonary effort or exertion, and the activity levels can include one or more of little effort, warm-up or recovery effort, aerobic effort, and anaerobic effort.

The plurality of activity levels can differ from one another in at least one activity parameters such as the activity intensity (I), the activity duration (σ), the activity transition pattern (δ), or any other descriptor of activity. In an example, the activity intensity can be measured using acceleration produced by an accelerometer sensor associated with a patient. The detected activity can be classified as vigorous exercise if the activity intensity equals or exceeds approximately 115 mG, and sustains for approximately 20 minutes or longer. The detected activity can be classified as moderate exercise if the activity intensity is between approximately 80 mG and 115 mG, and sustains for approximately 20 minutes or longer. Detected activities can be classified as mild exercise or activities of daily living if the activity intensity is less than 80 mG. In another example, classification of activity levels can be further based on the respective activity transition patterns, in addition to the activity intensity and the activity duration. The detected activity can be classified as vigorous exercise if the detected activity intensity parameter is within a first intensity zone (I_{V}), the activity duration parameter is within a first duration range (σ_{V}), and the activity transition pattern includes a first rate of change (δ_{V}) of activity intensity; or to classify the physical activity as moderate exercise when the detected activity intensity parameter is within a second intensity zone (I_{M}), the activity duration parameter is within a second duration range (σ_{M}), and the activity transition pattern includes a second rate of change (δ_{M}). The first intensity zone I_{V} includes activity intensity higher than that in the second intensity zone I_{M}, or the first duration range σ_{V} includes activity duration longer than that included in the second duration range σ_{M}, or the first rate of change δ_{V} is greater than the second rate of change δ_{M}.

The classification of the physical activity, such as produced at 630, can be used in different ways. In an example, the classified physical activities, either alone or in combination with other physiologic signals, can be used to generate diagnostic information about an existing disease or assessment of health condition. In another example, one or more parameters for therapy delivery, such as rate, frequency, duration, duty cycle, pulse width, pulse amplitude, or other parameters of electrostimulation, can be adjusted in accordance with the detected activity levels.

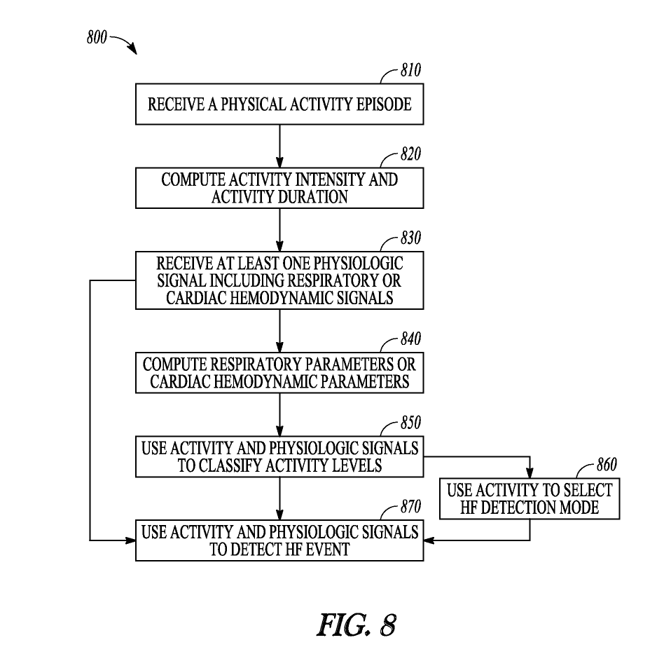

As illustrated in FIG. 6, the method 600 can optionally include one or more of operations at 640a, 640b, or 640c. At 640a, a heart failure (HF) event can be detected at least using the detected activity levels. Examples of the HF event can include HF decompensation event or comorbidities associated with HF, including renal insufficiency, diabetes mellitus, chronic obstructive pulmonary disease, sleeping disorders like obstructive and central apnea syndrome, anemia, among others. Examples of detecting a HF event using at least the detected activity levels are described below, such as with reference to FIG. 8.

At 640b, the classification of the physical activity can be used to stratify a patient's risk of developing a future event of worsening HF, such as a HF decompensation event in a specified timeframe (e.g., approximately 1-3 months, 3-6 months, or beyond 6 months). In an example, the risk, or the likelihood indication of a future HF event, can be inversely proportional to the frequency of the detected categorical activity level, such as moderate or vigorous exercise (F_{M} and F_{V}, respectively). The exercise frequency can be measured as the number of episodes of respective activity levels within a specified time period. In an example, a subject having an F_{M} exceeding a specified frequency threshold (e.g., 6 episodes per month), or an F_{V} exceeding a specified frequency threshold (e.g., 3 episodes per month), is deemed to have lower risk of developing future HF events than those whose F_{M} or F_{V} is below the specified frequency threshold.

The method 600 can additionally include receiving physiologic information such as obtained from one or more physiologic sensors. Examples of the physiologic information can include one or more of electrocardiogram, intracardiac electrogram, arrhythmia, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, body temperature, among other physiologic signals. In an example, the physiologic information can be presented to a system user at 640c along with the activity levels. In another example, the physiologic information can be used tougher with the detected activity level in detecting the HF event at 640a, or in stratifying the risk of future HF event at 640b.

Additionally or alternatively, a human-perceptible presentation of the detected one or more activity parameters, detected activity level, and the classification results can be generated at 640c. The human-perceptible presentation can also include a trend of activity levels or of other physical or physiologic measurements, summaries or statistics produced using the subject's historical activity levels or other physiologic measurements, or a comparison of the detected activity levels with a predetermined target exercise levels (e.g., exercise duration, intensity, frequency, etc.). The human-perceptible presentation can be delivered to a system user (e.g., a healthcare professional or a patient) such as via a user interface implemented in the external system 120. The presentation can include audio, text, graph, animation, or other audio-visual media formats that can inform, alert, or alarm the system user of the detected physical activity. In another example, the classified activity level and the detected one or more activity parameters can be transmitted via a wired or wireless communication network to a portable electronic device such as a handheld or wearable mobile communication device. The transmission can be triggered automatically on a scheduled or periodic basis (e.g., every 10-30 minutes, every hour, every day, every week, every month, or at any specified period), or it can be triggered manually such as in response to a command signal provided by the system user or a specified event.

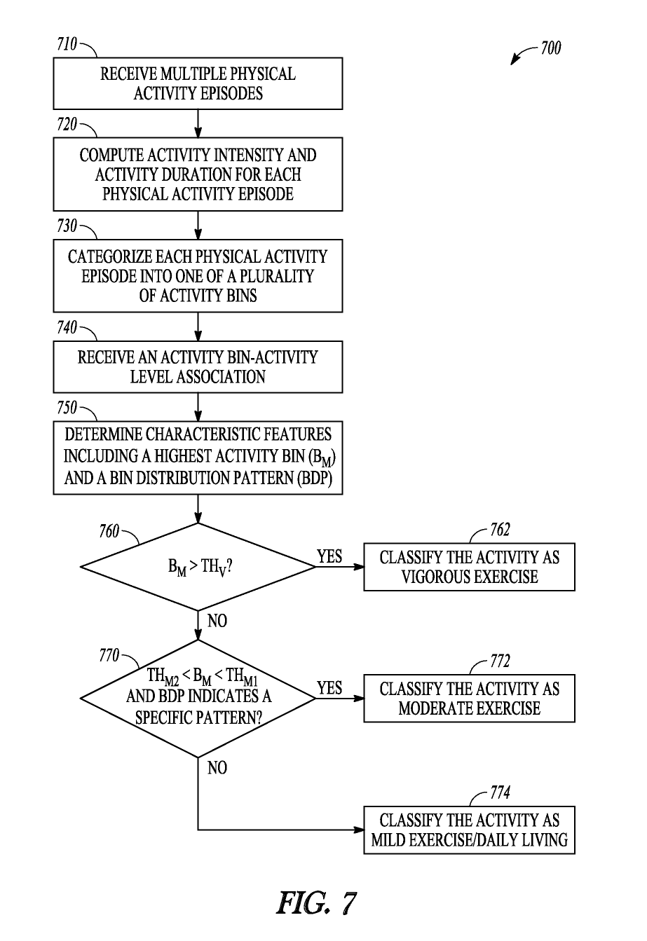

FIG. 7 illustrates an example of a method 700 for classifying physical activity based on categorization of physical activities. The method 700 can be an embodiment of the method 600, and can be performed by the physical activity detection and analyzer system 200 or any modification thereof.

The method 700 can begin at 710 where multiple activity episodes are received. The multiple activity episodes can be obtained during a specified period of time, such as during a day. The multiple activity episodes can be obtained from one sustained exercise session. At 720, activity parameters, including activity intensity and activity duration, can be computed for each physical activity episode.

At 730, each activity episode can be categorized into one of the plurality of bins {Bᵢ} (for i=1, 2, . . . , K) at least based on the one or more activity parameters obtained from the respective activity episode, where “i” denotes bin index and K denotes the highest possible bin number indicating the highest level of activity. An individual bin (Bᵢ) can include a respective bin count (NO indicating number of activity episodes categorized into the respective bin Bᵢ. A higher activity bin can be characterized by higher activity intensity or longer activity duration than a lower activity bin. In an example, the activity intensity can be measured as acceleration sensed by an accelerometer during exercise. The activity bin Bᵢ can be defined at least by a respective intensity range between Xᵢ mG to Yᵢ mG, where mG indicates unit of acceleration. A detected activity episode with intensity between Xᵢ mG and Yᵢ mG, and a corresponding activity duration exceeding a specified duration threshold (e.g., 10 minutes or 20 minutes), can be categorized into activity bin Bᵢ.

At 740, an activity bin—activity level association can be received such as from a memory device. The activity bin—activity level association can be constructed as a lookup table or other data structure, which establishes an association between an activity bin and an activity level (such as moderate or vigorous exercises). In an example, activity bins B₁₃ and above can be mapped to vigorous exercise, activity bins B₇ through B₁₂ can be mapped to moderate exercise, and activity bins B₂ through B₆ can be mapped to mild exercise or activities of daily living.

At 750, an activity bin distribution indicating spreading of bin counts across M bins {Bᵢ} (for i=1, 2, . . . , M, and M≦K) can be generated, where B_{M} is the highest activity bin that contains the activity episode with the highest activity level among the multiple activity. In an example, the activity bin distribution can include a histogram of M activity bins. One or more characteristic features can be determined from the activity bin distribution, including the bin index (M) of the highest activity bin B_{M}, and a bin distribution pattern indicative of relative bin counts of the plurality of activity bins from the activity bin distribution.

At 760, the highest activity bin B_{M} of the detected activity can be compared to a bin threshold for vigorous exercise, TH_{V}. An example of TH_{V} is B₁₃, which corresponds to acceleration within the range of approximately 134 mG to 145 mG). If B_{M} exceeds TH_{V}, then at 762 the detected activity can be classified as vigorous exercise. If B_{M} does not exceed TH_{V}, then at 770 B_{M} can be compared to bin thresholds (TH_{M1} and TH_{M2}) for moderate exercise. The bin distribution pattern can also be analyzed to determine if it manifests a specified pattern. Moderate exercise can have a characteristic activity transition pattern including an abrupt transition from a lower activity level to a higher activity level. If B_{M} falls in between TH_{M1} and TH_{M2}, and if the bin distribution pattern indicates that B_{M} is preceded by a specified number of lower activity bins with respective bin count below a specified bin count threshold value, the detected activity can then be classified as moderate exercise at 772. For example, the bin thresholds for moderate exercise are TH_{M1}=B₁₂ (approximately in a range between 120 mG to 132 mG) and THM2=B₇ (approximately in a range between 66 mG to 78 mG), and the bin distribution pattern is such that the activity bin B_{M} is separated from lower bins by at least two bins with zero bin counts. Such pattern suggests that the “transitional” physical activity prior to B_{M} does not sustain enough to be counted into one of the transitional bins. If, however, the conditions at 770 are not met, then at 774 the detected activity can be classified as mild exercise or activities of daily living.

FIG. 8 illustrates an example of a method 800 for detecting a heart failure (HF) event using at least physical activity level information. The method 800 can be an embodiment of the method 600, and can be performed by the physical activity detection and analyzer system 200 or any modification thereof.

A physical activity episode can be received at 810, and one or more activity parameters, including activity intensity and activity duration parameters can be computed at 820. At 830, at least one physiologic signal can be received. In an example, the physiologic signal can include a respiratory signal such as sensed using an impedance or air-flow sensors. In another example, the physiologic signal can include a cardiac hemodynamic signal such as sensed using a hemodynamic sensor. Examples of the hemodynamic sensors can include heart rate sensor, a pressure sensor configured for sensing arterial pressure, pulmonary artery pressure, left atrial pressure, RV pressure, LV coronary pressure; impedance sensors configured for sensing thoracic impedance or cardiac impedance; a temperature sensor configured for sensing blood temperature, an accelerometer or a microphone configured for sensing one or more heart sounds, an optical sensor such as a pulse oximeter configured for sensing blood oxygen saturation, a chemical sensor configured for sensing central venous pH value, or oxygen or carbon dioxide level in the blood or other tissues or organs.

At 840, respiratory parameters or cardiac hemodynamic parameters can be respectively computed from the respiratory or cardiac hemodynamic signals. Examples of the respiratory parameters can include a respiration rate, tidal volume, minute ventilation, peak or trough of a respiration signal, or other indicators of respiration depth; descriptors of respiration pattern such as apnea index indicating the frequency of sleep apnea, hypopnea index indicating the frequency of sleep hypopnea, apnea-hypopnea index (AHI) indicating the frequency of or sleep hypopnea events, or a rapid shallow breathing index (RSBI) computed as a ratio of respiratory frequency (number of breaths per minutes) to tidal volume. Examples of the hemodynamic parameters can include S1, S2, S3, or S4 heart sound components from the sensed heart sound signal, peak or trough impedance from the cardiac impedance signal, peak or trough blood pressure (corresponding respectively to systolic and diastolic pressures) from the blood pressure signal, or timing information associated with these signal components or characteristics.

At 850, one or more activity parameters and at least one physiologic parameter can be used to classify the physical activity into one of the plurality of activity levels. In an example, the physical activity can be classified as vigorous exercise when one or more detected activity intensity parameters fall within respective ranges (e.g., the activity intensity falls within a first intensity zone, or the activity duration parameter is within a first duration range), and at least one physiologic parameter meets a specified criterion (e.g., the heart rate (HR) exceeds a specified threshold or exceeds a baseline HR such as resting HR by a specified margin, or the respiration rate (RR) exceeds a specified threshold or exceeds a baseline RR such as resting RR by a specified margin). Likewise, the physical activity can be classified as moderate exercise when one or more detected activity intensity parameters fall within respective ranges different than those for vigorous exercise, and at least one physiologic parameter meets a specified criterion different than the criterion for vigorous exercise (e.g., a different HR threshold or RR threshold).

At 860, the classification of the activity levels can be used to select one of two or more HF detection modes. The HF detection modes can have different performances in detecting historical HF events. For example, a “high-sensitivity mode” can correspond to a higher sensitivity in detecting historical HF events, while a “low-sensitivity mode” can correspond to a lower sensitivity in detecting historical HF events. The HF detection modes can involve different algorithms, different threshold values for sensor responses, or different combinations or configurations of sensors used for detecting HF event. The present inventors have recognized that, compared to no exercise or lower-level exercise, sustained higher-level activities can reduce the likelihood of developing a HF event. As such, at 860, a “low-sensitivity mode” can be select in response to the physical activity being classified as vigorous exercise, while a “high-sensitivity mode” can be selected in response to the physical activity being classified as mild or moderate exercise.

At 870, the classified activity levels and the at least one physiologic signals can be used in detecting a HF event. A HF detection algorithm, such as that determined at 860 based on the activity level, can involve using the detected activity levels and one or more of a respiration rate (RR) signal, a tidal volume (TV) signal, or a heart rate (HR) signal to detect a HF event. In an example, a HF event can be detected in response to the physical activity being classified as a specified low-level exercise (e.g., mild exercise or activities of daily living) and the RR or TV exceeding a respective baseline value (RR₀ or TV₀ respectively, such as measured during a resting state) by a specified margin.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system, comprising:
a physical activity information receiver circuit, configured to receive information indicative of physical activity;
a physical activity analyzer circuit, coupled to the physical activity information receiver circuit, configured to detect one or more activity parameters using the physical activity information, and to classify the physical activity into one of a plurality of activity levels using the one or more activity parameters, the plurality of activity levels including two or more categorical activity levels;
wherein the one or more activity parameters includes an activity intensity parameter, an activity duration parameter, or an activity transition pattern including a change or a rate of change from a first activity level to a different second activity level.

2. The system of claim 1, wherein the plurality of activity levels includes two or more of a vigorous exercise, a moderate exercise, or a mild exercise or activities of daily living, and the physical activity analyzer circuit is configured to:
classify the physical activity as the vigorous exercise when the detected activity intensity parameter is within a first intensity zone and the activity duration parameter is within a first duration range; or
classify the physical activity as the moderate exercise when the detected activity intensity parameter is within a second intensity zone and the activity duration parameter is within a second duration range;
wherein the first intensity zone includes activity intensity higher than that included in the second intensity zone, or the first duration range includes activity duration longer than that included in the second duration range.

3. The system of claim 1, wherein the plurality of activity levels includes two or more of a vigorous exercise, a moderate exercise, or a mild exercise or activities of daily living, and the physical activity analyzer circuit is configured to:
classify the physical activity as the vigorous exercise when the detected activity intensity parameter is within a first intensity zone, the activity duration parameter is within a first duration range, and the activity transition pattern includes a first rate of change of activity intensity; or
classify the physical activity as the moderate exercise when the detected activity intensity parameter is within a second intensity zone, the activity duration parameter is within a second duration range, and the activity transition pattern includes a second rate of change of activity intensity;
wherein the first intensity zone includes activity intensity higher than that included in the second intensity zone, or the first duration range includes activity duration longer than that included in the second duration range, or the first rate of change is greater than the second rate of change.

4. The system of claim 1, comprising an activity categorizer circuit configured to categorize the detected one or more activity parameters into one of a plurality of activity bins;
wherein the physical activity analyzer circuit is configured to classify the physical activity using the categorization of the activity parameter and a bin-activity level association that corresponds the plurality of activity bins to one or more of the plurality of activity levels;
wherein an individual bin is characterized by a respective activity intensity zone and a respective activity duration range, a higher activity bin having higher activity intensity or longer activity duration than a lower activity bin.

5. The system of claim 4, wherein the physical activity information includes multiple activity episodes obtained during a specified period of time, and the activity categorizer circuit is further configured to:
generate an activity bin distribution using categorization of the multiple activity episodes, an individual bin including a bin count indicating number of activity episodes categorized into the respective bin; and
determine at least one characteristic feature from the activity bin distribution; and
wherein the physical activity analyzer circuit is configured to classify the physical activity using the at least one characteristic feature of the activity bin distribution.

6. The system of claim 5, wherein the activity categorizer circuit is configured to determine the at least one characteristic feature including a highest activity bin and a bin distribution pattern, the highest activity bin including an activity episode of highest activity intensity among the multiple activity episodes, the bin distribution pattern indicating a comparison of bin counts of the plurality of activity bins; and
wherein the plurality of activity levels includes two or more of a vigorous exercise, a moderate exercise, or a mild exercise or activities of daily living, and the physical activity analyzer circuit is configured to:
classify the physical activity as the vigorous exercise when the highest activity bin exceeds a first bin threshold; or
classify the physical activity as the moderate exercise when (1) the highest activity bin is between the first bin threshold and a second bin threshold lower than the first bin threshold, and (2) the bin distribution pattern indicates the highest activity bin preceded by a specified number of lower activity bins with respective bin count below a specified bin count threshold value.

7. The system of claim 1, comprising a physiologic signal receiver circuit configured to receive at least one physiologic signal obtained during the physical activity, the at least one physiologic signal including a respiratory signal or a cardiac hemodynamic signal,
wherein the physical activity analyzer circuit is configured to classify the physical activity using the one or more activity parameters and the at least one physiologic signal.

8. The system of claim 1, comprising an adjudication input circuit configured to receive adjudication or confirmation of the classified activity level, wherein the physical activity analyzer circuit is configured to classify the physical activity at least using the received adjudication.

9. The system of claim 1, comprising a heart failure (HF) detector circuit configured to determine an activity trend indicative of temporal variation of the classified activity level, and to detect a HF event indicative of worsening HF at least using the activity trend.

10. The system of claim 9, wherein the HF detector circuit is configured to operate in a first mode to detect the HF event in response to the physical activity being classified as a first activity level, but to operate in a different second mode to detect the HF event in response to the physical activity being classified as a second activity level more vigorous than the first activity level, wherein the HF detector, when operated in the first mode, has a higher sensitivity in detecting historical HF events than when operated in the second mode.

11. The system of claim 9, comprising a physiologic sensor circuit configured to sense at least one physiologic signal obtained during the physical activity, wherein the HF detector circuit is configured to detect the HF event using both the activity trend and the sensed physiologic signal.

12. The system of claim 1, comprising a heart failure (HF) stratifier circuit configured to determine an exercise frequency of the detected categorical activity level during a specified time, and to determine a likelihood indication of a future event of worsening HF, the likelihood indication inversely proportional to the exercise frequency.

13. The system of claim 1, comprising an output circuit configured to generate a human-perceptible presentation of information including the classified activity level and the detected one or more activity parameters.

14. A method for analyzing physical activity experienced by a patient using a medical apparatus, comprising:
receiving information of physical activity;
detecting one or more activity parameters using the physical activity information, the one or more activity parameters including an activity intensity parameter, an activity duration parameter, or an activity transition pattern including a change or a rate of change from a first activity level to a different second activity level; and
classifying the physical activity into one of a plurality of activity levels using the one or more activity parameters, the plurality of activity levels including two or more categorical activity levels.

15. The method of claim 14, wherein the plurality of activity levels includes two or more of a vigorous exercise, a moderate exercise, or a mild exercise or activities of daily living, and wherein classifying the physical activity includes:
classifying the physical activity as the vigorous exercise when the detected activity intensity parameter is within a first intensity zone, the activity duration parameter is within a first duration range, and the activity transition pattern includes a first rate of change of activity intensity meeting a specified criterion; or
classifying the physical activity as the moderate exercise when the detected activity intensity parameter is within a second intensity zone, and the activity duration parameter is within a second duration range;
wherein the first intensity zone has higher intensity than the second intensity zone, and the first duration range has longer duration than the second duration range.

16. The method of claim 14, comprising:
categorizing the detected one or more activity parameters into one of a plurality of activity bins; and
providing an activity bin-activity level association that corresponds the plurality of activity bins to one or more of the plurality of activity levels;
wherein classifying the physical activity includes classifying the physical activity using the categorization of the activity parameter and the bin-activity level association; and
wherein an individual bin is characterized by a respective activity intensity zone and a respective activity duration range, a higher activity bin having higher activity intensity or longer activity duration than a lower activity bin.

17. The method of claim 16, comprising:
generating an activity bin distribution using categorization of multiple activity episodes obtained during a specified period of time, an individual bin including a bin count indicating number of activity episodes categorized into the respective bin; and
determining at least one characteristic feature from the activity bin distribution;
wherein classifying the physical activity includes classifying the physical activity using the at least one characteristic feature of the activity bin distribution.

18. The method of claim 14, comprising receiving at least one physiologic signal obtained during the physical activity, the at least one physiologic signal including a respiratory signal or a cardiac hemodynamic signal,
wherein classifying the physical activity includes classifying the physical activity using the one or more activity parameters and the at least one physiologic signal.

19. The method of claim 14, comprising determining an activity trend indicative of temporal variation of the classified activity level, and detecting a heart failure (HF) event indicative of worsening HF at least using the activity trend.

20. The method of claim 19, comprising receiving at least one physiologic signal obtained during the physical activity, wherein detecting the HF event includes detecting the HF event when the detected activity trend and the at least one physiologic signal meet respective criteria.
